SEQUENCE LISTING (6 sequences)

SEQ ID NO: 1 - DNA, 2814 bp, synthetic construct
  source 1..2814: mol_type = other DNA
atgagcaccatgcgcctgctgacgctcgccctgctgttctcctgctccgtcgcccgtgccgcgtgcgaccccaagatcgtcaacattggcgcggtgctgagcacgcggaagcacgagcagatgttccgcgaggccgtgaaccaggccaacaagcggcacggctcctggaagattcagctcaatgccacctccgtcacgcacaagcccaacgccatccagatggctctgtcggtgtgcgaggacctcatctccagccaggtctacgccatcctagttagccatccacctacccccaacgaccacttcactcccacccctgtctcctacacagccggcttctaccgcatacccgtgctggggctgaccacccgcatgtccatctactcggacaagagcatccacctgagcttcctgcgcaccgtgccgccctactcccaccagtccagcgtgtggtttgagatgatgcgtgtctacagctggaaccacatcatcctgctggtcagcgacgaccacgagggccgggcggctcagaaacgcctggagacgctgctggaggagcgtgagtccaaggcagagaaggtgctgcagtttgacccagggaccaagaacgtgacggccctgctgatggaggcgaaagagctggaggcccgggtcatcatcctttctgccagcgaggacgatgctgccactgtataccgcgcagccgcgatgctgaacatgacgggctccgggtacgtgtggctggtcggcgagcgcgagatctcggggaacgccctgcgctacgccccagacggcatcctcgggctgcagctcatcaacggcaagaacgagtcggcccacatcagcgacgccgtgggcgtggtggcccaggccgtgcacgagctcctcgagaaggagaacatcaccgacccgccgcggggctgcgtgggcaacaccaacatctggaagaccgggccgctcttcaagagagtgctgatgtcttccaagtatgcggatggggtgactggtcgcgtggagttcaatgaggatggggaccggaagttcgccaactacagcatcatgaacctgcagaaccgcaagctggtgcaagtgggcatctacaatggcacccacgtcatccctaatgacaggaagatcatctggccaggcggagagacagagaagcctcgagggtaccagatgtccaccagactgaagattgtgacgatccaccaggagcccttcgtgtacgtcaagcccacgctgagtgatgggacatgcaaggaggagttcacagtcaacggcgacccagtcaagaaggtgatctgcaccgggcccaacgacacgtcgccgggcagcccccgccacacggtgcctcagtgttgctacggcttttgcatcgacctgctcatcaagctggcacggaccatgaacttcacctacgaggtgcacctggtggcagatggcaagttcggcacacaggagcgggtgaacaacagcaacaagaaggagtggaatgggatgatgggcgagctgctcagcgggcaggcagacatgatcgtggcgccgctaaccataaacaacgagcgcgcgcagtacatcgagttttccaagcccttcaagtaccagggcctgactattctggtcaagaaggagattccccggagcacgctggactcgttcatgcagccgttccagagcacactgtggctgctggtggggctgtcggtgcacgtggtggccgtgatgctgtacctgctggaccgcttcagccccttcggccggttcaaggtgaacagcgaggaggaggaggaggacgcactgaccctgtcctcggccatgtggttctcctggggcgtcctgctcaactccggcatcggggaaggcgcccccagaagcttctcagcgcgcatcctgggcatggtgtgggccggctttgccatgatcatcgtggcctcctacaccgccaacctggcggccttcctggtgctggaccggccggaggagcgcatcacgggcatcaacgaccctcggctgaggaacccctcggacaagtttatctacgccacggtgaagcagagctccgtggatatctacttccggcgccaggtggagctgagcaccatgtaccggcatatggagaagcacaactacgagagtgcggcggaggccatccaggccgtgagagacaacaagctgcatgccttcatctgggactcggcggtgctggagttcgaggcctcgcagaagtgcgacctggtgacgactggagagctgtttttccgctcgggcttcggcataggcatgcgcaaagacagcccctggaagcagaacgtctccctgtccatcctcaagtcccacgagaatggcttcatggaagacctggacaagacgtgggttcggtatcaggaatgtgactcgcgcagcaacgcccctgcgacccttacttttgagaacatggccggggtcttcatgctggtagctgggggcatcgtggccgggatcttcctgattttcatcgagattgcctacaagcggcacaaggatgctcgccggaagcagatgcagctggcctttgccgccgttaacgtgtggcggaagaacctgcaggatagaaagagtggtagagcagagcctgaccctaaaaagaaagccacatttagggctatcacctccaccctggcttccagcttcaagaggcgtaggtcctccaaagacacgagcaccgggggtggacgcggcgctttgcaaaaccaaaaagacacagtgctgccgcgacgcgctattgagagggaggagggccagctgcagctgtgttcccgtcatagggagagc

SEQ ID NO: 2 - DNA, 2697 bp, synthetic construct
  source 1..2697: mol_type = other DNA
atgttgctgctgctgctactggcgccactcttcctccgccccccgggcgcgggcggggcgcagacccccaacgccacctcagaaggttgccagatcatacacccgccctgggaagggggcatcaggtaccggggcctgactcgggaccaggtgaaggctatcaacttcctgccagtggactatgagattgagtatgtgtgccggggggagcgcgaggtggtggggcccaaggtccgcaagtgcctggccaacggctcctggacagatatggacacacccagccgctgtgtgaatcgaacgccacactcagaacggcgcgcagtgtacatcggggcactgtttcccatgagcgggggctggccagggggccaggcctgccagcccgcggtggagatggcgctggaggacgtgaatagccgcagggacatcctgccggactatgagctcaagctcatccaccacgacagcaagtgtgatccaggccaagccaccaagtacctatatgagctgctctacaacgaccctatcaagatcatccttatgcctggctgcagctctgtctccacgctggtggctgaggctgctaggatgtggaacctcattgtgctttcctatggctccagctcaccagccctgtcaaaccggcagcgtttccccactttcttccgaacgcacccatcagccacactccacaaccctacccgcgtgaaactctttgaaaagtggggctggaagaagattgctaccatccagcagaccactgaggtcttcacttcgactctggacgacctggaggaacgagtgaaggaggctggaattgagattactttccgccagagtttcttctcagatccagctgtgcccgtcaaaaacctgaagcgccaggatgcccgaatcatcgtgggacttttctatgagactgaagcccggaaagttttttgtgaggtgtacaaggagcgtctctttgggaagaagtacgtctggttcctcattgggtggtatgctgacaattggttcaagatctacgacccttctatcaactgcacagtggatgagatgactgaggcggtggagggccacatcacaactgagattgtcatgctgaatcctgccaatacccgcagcatttccaacatgacatcccaggaatttgtggagaaactaaccaagcgactgaaaagacaccctgaggagacaggaggcttccaggaggcaccgctggcctatgatgccatctgggccttggcactggccctgaacaagacatctggaggaggcggccgttctggtgtgcgcctggaggacttcaactacaacaaccagaccattaccgaccaaatctaccgggcaatgaactcttcgtcctttgagggtgtctctggccatgtggtgtttgatgccagcggctctcggatggcatggacgcttatcgagcagcttcagggtggcagctacaagaagattggctactatgacagcaccaaggatgatctttcctggtccaaaacagataaatggattggagggtcccccccagctgaccagaccctggtcatcaagacattccgcttcctgtcacagaaactctttatctccgtctcagttctctccagcctgggcattgtcctagctgttgtctgtctgtcctttaacatctacaactcacatgtccgttatatccagaactcacagcccaacctgaacaacctgactgctgtgggctgctcactggctttagctgctgtcttccccctggggctcgatggttaccacattgggaggaaccagttccctttcgtctgccaggcccgcctctggctcctgggcctgggctttagtctgggctacggttccatgttcaccaagatttggtgggtccacacggtcttcacaaagaaggaagaaaagaaggagtggaggaagactctggaaccctggaagctgtatgccacagtgggcctgctggtgggcatggatgtcctcactctcgccatctggcagatcgtggaccctctgcaccggaccattgagacatttgccaaggaggaacctaaggaagatattgacgtctctattctgccccagctggagcattgcagctccaggaagatgaatacatggcttggcattttctatggttacaaggggctgctgctgctgctgggaatcttccttgcttatgagaccaagagtgtgtccactgagaagatcaatgatcaccgggctgtgggcatggctatctacaatgtggcagtcctgtgcctcatcactgctcctgtcaccatgattctgtccagccagcaggatgcagcctttgcctttgcctctcttgccatagttttctcctcctatatcactcttgttgtgctctttgtgcccaagatgcgcaggctgatcacccgaggggaatggcagtcggaggcgcaggacaccatgaagacagggtcatcgaccaacaacaacgaggaggagaagtcccggctgttggagaaggagaaccgtgaactggaaaagatcattgctgagaaagaggagcgtgtctctgaactgcgccatcaactccagtctcggcagcagctccgctcccggcgccacccaccgacacccccagaaccctctgggggcctgcccaggggaccccctgagccccccgaccggcttagctgtgatgggagtcgagtgcatttgctttataag

SEQ ID NO: 3 - DNA, 2718 bp, synthetic construct
  source 1..2718: mol_type = other DNA
atgcagcacatttttgccttcttctgcaccggtttcctaggcgcggtagtaggtgccaatttccccaacaatatccagatcgggggattatttccaaaccagcagtcacaggaacatgctgcttttagatttgctttgtcgcaactcacagagcccccgaagctgctcccccagattgatattgtgaacatcagcgacagctttgagatgacctatagattctgttcccagttctccaaaggagtctatgccatctttgggttttatgaacgtaggactgtcaacatgctgacctccttttgtggggccctccacgtctgcttcattacgccgagctttcccgttgatacatccaatcagtttgtccttcagctgcgccctgaactgcaggatgccctcatcagcatcattgaccattacaagtggcagaaatttgtctacatttatgatgccgaccggggcttatccgtcctgcagaaagtcctggatacagctgctgagaagaactggcaggtgacagcagtcaacattttgacaaccacagaggagggataccggatgctctttcaggacctggagaagaaaaaggagcggctggtggtggtggactgtgaatcagaacgcctcaatgctatcttgggccagattataaagctagagaagaatggcatcggctaccactacattcttgcaaatctgggcttcatggacattgacttaaacaaattcaaggagagtggcgccaatgtgacaggtttccagctggtgaactacacagacactattccggccaagatcatgcagcagtggaagaatagtgatgctcgagaccacacacgggtggactggaagagacccaagtacacctctgcgctcacctacgatggggtgaaggtgatggctgaggctttccagagcctgcggaggcagagaattgatatatctcgccgggggaatgctggggattgtctggctaacccagctgttccctggggccaagggatcgacatccagagagctctgcagcaggtgcgatttgaaggtttaacaggaaacgtgcagtttaatgagaaaggacgccggaccaactacacgctccacgtgattgaaatgaaacatgacggcatccgaaagattggttactggaatgaagatgataagtttgtccctgcagccaccgatgcccaagctgggggcgataattcaagtgttcagaacagaacatacatcgtcacaacaatcctagaagatccttatgtgatgctcaagaagaacgccaatcagtttgagggcaatgaccgttacgagggctactgtgtagagctggcggcagagattgccaagcacgtgggctactcctaccgtctggagattgtcagtgatggaaaatacggagcccgagaccctgacacgaaggcctggaatggcatggtgggagagctggtctatggaagagcagatgtggctgtggctcccttaactatcactttggtccgggaagaagttatagatttctccaaaccatttatgagtttggggatctccatcatgattaaaaaaccacagaaatccaagccgggtgtcttctccttccttgatcctttggcttatgagatttggatgtgcattgtttttgcctacattggagtgagtgttgtcctcttcctggtcagccgcttcagtccctatgaatggcacagtgaagagtttgaggaaggacgggaccagacaaccagtgaccagtccaatgagtttgggatattcaacagtttgtggttctccctgggagccttcatgcagcaaggatgtgacatttctcccaggtccctgtctggtcgcatcgttggtggcgtctggtggttcttcaccttaatcatcatctcctcatatacagccaatctggccgccttcctgaccgtggagaggatggtgtctcccattgagagtgcagaggacctagcgaagcagacagaaattgcctacgggacgctggaagcaggatctactaaggagttcttcaggaggtctaaaattgctgtgtttgagaagatgtggacatacatgaagtcagcagagccatcagtttttgtgcggaccacagaggaggggatgattcgagtgaggaaatccaaaggcaaatatgcctacctcctggagtccaccatgaatgagtacattgagcagcggaaaccctgtgacaccatgaaggtgggaggtaacttggattccaaaggctatggcattgcaacacccaaggggtctgccctgagaaatccagtaaacctggcagtgttaaaactgaacgagcaggggcttttggacaaattgaaaaacaaatggtggtacgacaagggcgagtgcggcagcgggggaggtgattccaaggacaagacaagcgctctgagcctcagcaatgtggcaggcgtgttctacatcctgatcggaggacttggactagccatgctggttgccttaatcgagttctgctacaaatcccgtagtgaatccaagcggatgaagggtttttgtttgatcccacagcaatccatcaacgaagccatacggacatcgaccctcccccgcaacagcggggcaggagccagcagcggcggcagtggagagaatggtcgggtggtcagccatgacttccccaagtccatgcaatcgattccttgcatgagccacagttcagggatgcccttgggagccacgggattg

SEQ ID NO: 4 - DNA, 3993 bp, synthetic construct
  source 1..3993: mol_type = other DNA
atgcaggcggctccgcgcgccggctgcggggcagcgctcctgctgtggattgtcagcagctgcctctgcagagcctggacggctccctccacgtcccaaaaatgtgatgagccacttgtctctggactcccccatgtggctttcagcagctcctcctccatctctggtagctattctcccggctatgccaagataaacaagagaggaggtgctgggggatggtctccatcagacagcgaccattatcaatggcttcaggttgactttggcaatcggaagcagatcagtgccattgcaacccaaggaaggtatagcagctcagattgggtgacccaataccggatgctctacagcgacacagggagaaactggaaaccctatcatcaagatgggaatatctgggcatttcccggaaacattaactctgacggtgtggtccggcacgaattacagcatccgattattgcccgctatgtgcgcatagtgcctctggattggaatggagaaggtcgcattggactcagaattgaagtttatggctgttcttactgggctgatgttatcaactttgatggccatgttgtattaccatatagattcagaaacaagaagatgaaaacactgaaagatgtcattgccttgaactttaagacgtctgaaagtgaaggagtaatcctgcacggagaaggacagcaaggagattacattaccttggaactgaaaaaagccaagctggtcctcagtttaaacttaggaagcaaccagcttggccccatatatggccacacatcagtgatgacaggaagtttgctggatgaccaccactggcactctgtggtcattgagcgccaggggcggagcattaacctcactctggacaggagcatgcagcacttccgtaccaatggagagtttgactacctggacttggactatgagataacctttggaggcatccctttctctggcaagcccagctccagcagtagaaagaatttcaaaggctgcatggaaagcatcaactacaatggcgtcaacattactgatcttgccagaaggaagaaattagagccctcaaatgtgggaaatttgagcttttcttgtgtggaaccctatacggtgcctgtctttttcaacgctacaagttacctggaggtgcccggacggcttaaccaggacctgttctcagtcagtttccagtttaggacatggaaccccaatggtctcctggtcttcagtcactttgcggataatttgggcaatgtggagattgacctcactgaaagcaaagtgggtgttcacatcaacatcacacagaccaagatgagccaaatcgatatttcctcaggttctgggttgaatgatggacagtggcacgaggttcgcttcctagccaaggaaaattttgctattctcaccatcgatggagatgaagcatcagcagttcgaactaatagtccccttcaagttaaaactggcgagaagtacttttttggaggttttctgaaccagatgaataactcaagtcactctgtccttcagccttcattccaaggatgcatgcagctcattcaagtggacgatcaacttgtaaatttatacgaagtggcacaaaggaagccgggaagtttcgcgaatgtcagcattgacatgtgtgcaatcatagacagatgtgtgcccaatcactgtgagcatggtggaaagtgctcgcaaacatgggacagcttcaaatgcacttgtgatgagacaggatacagtggggccacctgccacaactctatctacgagccttcctgtgaagcctacaaacacctaggacagacatcaaattattactggatagatcctgatggcagcggacctctggggcctctgaaagtttactgcaacatgacagaggacaaagtgtggaccatagtgtctcatgacttgcagatgcagacgcctgtggtcggctacaacccagaaaaatactcagtgacacagctcgtttacagcgcctccatggaccagataagtgccatcactgacagtgccgagtactgcgagcagtatgtctcctatttctgcaagatgtcaagattgttgaacaccccagatggaagcccttacacttggtgggttggcaaagccaacgagaagcactactactggggaggctctgggcctggaatccagaaatgtgcctgcggcatcgaacgcaactgcacagatcccaagtactactgtaactgcgacgcggactacaagcaatggaggaaggatgctggtttcttatcatacaaagatcacctgccagtgagccaagtggtggttggagatactgaccgtcaaggctcagaagccaaattgagcgtaggtcctctgcgctgccaaggagacaggaattattggaatgccgcctctttcccaaacccatcctcctacctgcacttctctactttccaaggggaaactagcgctgacatttctttctacttcaaaacattaaccccctggggagtgtttcttgaaaatatgggaaaggaagatttcatcaagctggagctgaagtctgccacagaagtgtccttttcatttgatgtgggaaatgggccagtagagattgtagtgaggtcaccaacccctctcaacgatgaccagtggcaccgggtcactgcagagaggaatgtcaagcaggccagcctacaggtggaccggctaccgcagcagatccgcaaggccccaacagaaggccacacccgcctggagctctacagccagttatttgtgggtggtgctgggggccagcagggcttcctgggttgcatccgctccttgaggatgaatggggtgacacttgacctggaggaaagagcaaaggtcacatctgggttcatatccggatgctcgggccattgcaccagctatggaacaaactgtgaaaatggaggcaaatgcctagagagataccacggttactcctgcgattgctctaatactgcatatgatggaacattttgcaacaaagatgttggtgcattttttgaagaagggatgtggctacgatataactttcaggcaccagcaacaaatgccagagactccagcagcagagtagacaacgctcccgaccagcagaactcccacccggacctggcacaggaggagatccgcttcagcttcagcaccaccaaggcgccctgcattctcctctacatcagctccttcaccacagacttcttggcagtcctcgtcaaacccactggaagcttacagattcgatacaacctgggtggcacccgagagccatacaatattgacgtagaccacaggaacatggccaatggacagccccacagtgtcaacatcacccgccacgagaagaccatctttctcaagctcgatcattatccttctgtgagttaccatctgccaagttcatccgacaccctcttcaattctcccaagtcgctctttctgggaaaagttatagaaacagggaaaattgaccaagagattcacaaatacaacaccccaggattcactggttgcctctccagagtccagttcaaccagatcgcccctctcaaggccgccttgaggcagacaaacgcctcggctcacgtccacatccagggcgagctggtggagtccaactgcggggcctcgccgctgaccctctcccccatgtcgtccgccaccgacccctggcacctggatcacctggattcagccagtgcggattttccatataatccaggacaaggccaagctataagaaatggagtcaacagaaactcggctatcattggaggcgtcattgctgtggtgattttcaccatcctgtgcaccctggtcttcctgatccggtacatgttccgccacaagggcacctaccataccaacgaagcaaagggggcggagtcggcagagagcgcggacgccgccatcatgaacaacgaccccaacttcacagagaccattgatgaaagcaaaaaggaatggctcatt

SEQ ID NO: 5 - DNA, 1671 bp, synthetic construct
  source 1..1671: mol_type = other DNA
atggaatcagaaagaagcaaaaggatgggaaatgcctgcattcccctgaaaagaattgcttatttcctatgtctcttatctgcgcttttgctgactgaggggaagaaaccagcgaagccaaaatgccctgccgtgtgtacttgtaccaaagataatgctttatgtgagaatgccagatccattccacgcaccgttcctcctgatgttatctcattatcctttgtgagatctggttttactgaaatctcagaagggagttttttattcacgccatcgctgcagctcttgttattcacatcgaactcctttgatgtgatcagtgatgatgcttttattggtcttccacatctagagtatttattcatagaaaacaacaacatcaagtcaatttcaagacatactttccggggactaaagtcattaattcacttgagccttgcaaacaacaatctccagacactcccaaaagatattttcaaaggcctggattctttaacaaatgtggacctgaggggtaattcatttaattgtgactgtaaactgaaatggctagtggaatggcttggccacaccaatgcaactgttgaagacatctactgcgaaggccccccagaatacaagaagcgcaaaatcaatagtctctcctcgaaggatttcgattgcatcattacagaatttgcaaagtctcaagacctgccttatcaatcattgtccatagacactttttcttatttgaatgatgagtatgtagtcatcgctcagccttttactggaaaatgcattttccttgaatgggaccatgtggaaaagaccttccggaattatgacaacattacaggcacatccactgtagtatgcaagcctatagtcattgaaactcagctctatgttattgtggcccagctgtttggtggctctcacatctataagcgagacagttttgcaaataaattcataaaaatccaggatattgaaattctcaaaatccgaaaacccaatgacattgaaacattcaagattgaaaacaactggtactttgttgttgctgacagttcaaaagctggttttactaccatttacaaatggaacggaaacggattctactcccatcaatccttacacgcgtggtacagggacactgatgtggaatatctagaaatagtcagaacacctcagacactcagaacgcctcatttaattctgtctagtagttcccagcgtcctgtaatttatcagtggaacaaagcaacacaattattcactaaccaaactgacattcctaacatggaggatgtgtacgcagtgaagcacttctcagtgaaaggggacgtgtacatttgcttgacaagattcattggtgattccaaagtcatgaaatggggaggctcctcgttccaggatattcagaggatgccatcgcgaggatccatggtgttccagcctcttcaaataaataattaccaatatgcaattcttggaagtgattactcctttactcaagtgtataactgggatgcagagaaagccaaatttgtgaaatttcaggaattaaatgttcaggcaccaagatcattcacacatgtgtccattaataagcgtaattttctttttgcttccagttttaagggaaatacacagatttacaaacatgtcatagttgacttaagcgca

SEQ ID NO: 6 - DNA, 2403 bp, synthetic construct
  source 1..2403: mol_type = other DNA
atgaaggaaaaggccatgatcaagaccgctaagatgcaggggaacgtgatggagctggtggggagtaaccctccgcagaggaattggaaaggaatagcaattgcactgcttgtcattctggtcatctgctccttgatcgtcacctcggtcatacttctgacaccagcggaagataatagtctgtctcaaaagaagaaggtcactgtagaagatctcttcagtgaagacttcaaaattcatgaccccgaggctaagtggataagtgatacagaattcatctacagagaacagaaaggaacagtgagactgtggaatgttgaaacaaatacttctactgtcttaatagaaggcaaaaaaattgaatcattaagagccatcagatatgaaatatctccagatagagagtatgcacttttttcatacaatgtggaacccatatatcaacactcgtatactggatattatgtcctgagcaaaattcctcatggggatcctcaaagtctggacccaccagaagtcagcaatgcaaaacttcagtatgcaggatggggccctaaaggccaacagctgatatttatttttgaaaacaatatctactactgtgcacatgtcgggaaacaggccatccgtgtggtctccactggcaaggaaggtgtgatttacaatggcctcagtgactggctgtatgaagaggagattttgaagacacacatcgcacactggtggtctccggatggcacgagactcgcctacgccgccatcaatgattcccgtgtccccatcatggagctcccaacttacaccggctccatctaccccaccgtgaagccctaccactatcccaaggctggaagtgagaaccccagcatttccctacacgttattggcttaaatggacccacccatgatctggagatgatgccgcctgatgatccacggatgagggagtactacatcaccatggtgaagtgggccaccagcaccaaggtcgccgtgacctggctgaaccgggcgcagaacgtgtccatcctcaccctctgcgacgccaccacgggggtctgcacgaagaaacacgaggatgaaagtgaggcctggctccacagacagaatgaagaacctgtgttctccaaggatggccgaaagtttttcttcatcagagccatcccccagggaggacgagggaaattctatcacatcacggtgtcctcgtcccagcccaacagcagcaacgacaacatccagtccatcacctccggggactgggacgtgaccaagatcctagcctacgatgagaaggggaataagatctacttcctgagcacggaggacctgcctcggagacgacaactctacagtgccaacacggtgggcaacttcaacaggcagtgcctctcctgtgacctggttgagaactgcacctacttcagcgcttccttcagccatagcatggacttcttcctgctcaagtgcgaaggtcctggtgttcctatggtgacggtgcacaacacaacagataagaaaaaaatgtttgacctagaaacaaatgaacatgtcaagaaggccataaatgaccgacagatgcctaaagtggaatacagggacattgagattgatgattacaacctgcccatgcagatactgaagccagcaaccttcaccgacaccacccactaccctctgctcctggtggtggatggcaccccgggcagccagagtgtggctgagaagttcgaggtgagctgggagacggtgatggtgagcagccacggcgcggtggtggtaaagtgtgacggccgtggcagcggcttccaagggaccaagctcctgcacgaagtgaggcggcggctgggcttgctggaggagaaggaccagatggaggccgtgcggacgatgctgaaggagcagtacattgacaggacgcgcgtggccgtgtttgggaaggattacggtggctacctgagcacctacatcctcccagcaaagggagaaaatcaaggccagacattcacctgcggctctgctctctctccaataacagacttcaaactctatgcctctgcgttttccgagaggtacttgggcctccatggacttgacaacagagcatacgagatgaccaaggtagcccatcgagtctccgcgctggaagaacagcagttcctgatcattcatcccactgccgatgaaaaaattcatttccagcacacagcagaactcattacacaactaattaggggaaaggctaattacagcttacagatttacccggacgaaagccattactttaccagctccagcctcaaacagcatctgtaccggtccatcatcaacttcttcgtggaatgcttcaggatccaggacaaactgctgacagtcacagcgaaagaggacgaggaggaggac

(19) United States
(12) Patent Application Publication
(10) Pub. No.: US 2026/0276635 A1
(43) Pub. Date: Sep. 17, 2026

(54) METHODS OF DETECTING AND QUANTIFYING AUTOANTIBODIES AGAINST NEURONAL ANTIGENS

(71) Applicant: UNI PHARMA CO., LTD., Taipei (TW)

(72) Inventors: Yu-Shan WANG, Taipei City (TW); Ting-En LIAO, Taipei City (TW); Pei-Cheng CHIU, Taipei City (TW); Shan-Shan KUO, Taipei City (TW); Szu-Ting CHEN, Taipei City (TW); Chih-Wei CHAO, Taipei City (TW)

(21) Appl. No.: 19/570,075

(22) Filed: Mar. 17, 2026

Related U.S. Application Data
(60) Provisional application No. 63/773,203, filed on Mar. 17, 2025.

(51) Int. Cl. G01N 33/564; G01N 21/64; G01N 33/58

(52) U.S. Cl. CPC G01N 33/564; G01N 21/6428; G01N 33/582; G01N 2021/6439; G01N 2333/705; G01N 2333/70571; G01N 2800/28; G01N 2800/52

(57) ABSTRACT

Disclosed herein is a method of identifying and treating a subject having autoimmune encephalitis (AE). The method includes steps of (1) detecting an autoantibody against a neuronal antigen in a biological sample obtained from the subject, in which the neuronal antigen may be N-methyl-D-aspartate receptor (NMDAR), α-amino-3-hydroxy-5-methyl-4-isoxazoleproprionic acid receptor (AMPAR), gamma-aminobutyric acid receptor (GABAR), contactin-associated protein-like 2 (Caspr2), leucine-rich glioma inactivated protein 1 (LGI1), or dipeptidyl-peptidase-like protein 6 (DPPX); and (2) treating the subject by administering solumedrol or immunoglobulin (Ig) to the subject to ameliorate symptoms associated with AE.

### REFERENCE TO A SEQUENCE LISTING

The present application is being filed along with a Sequence Listing in electronic format. The Sequence Listing is provided as a file entitled “P4436-US_SeqList”, created on Mar. 12, 2026, which is 22,399 bytes in size. The information in the electronic format of the Sequence Listing is incorporated herein by reference in its entirety

### CROSS-REFERENCES TO RELATED APPLICATIONS

This application claims priority and the benefit of U.S. Provisional Patent Application No. 63/773,203, filed Mar. 17, 2025, the entirety of which is incorporated herein by reference.

### BACKGROUND OF THE INVENTION

### 1. Field of the Invention

The present disclosure relates to methods of detecting and quantifying autoantibodies specifically targeting a neuronal antigen associated with autoimmune encephalitis (AE).

### 2. Description of Related Art

Autoimmune encephalitis (AE) refers to a type of brain inflammation where the body's immune system (e.g., antibodies) attacks healthy cells and tissues in the brain and/or spinal cord. It is a rare and complex disease that may cause rapid changes in both physical and mental health. Common symptoms of AE include impaired memory, abnormal or involuntary movements, difficulty with balance, speech or vision, insomnia, seizures, weakness, numbness, severe anxiety, compulsive behaviors, hallucinations, delusions, loss of consciousness, and coma.

Depending on the neuronal protein attacked by the antibody (also known as “anti-neuronal antibody”), AE may be divided into different types, including anti-N-methyl-D-aspartate receptor (NMDAR) encephalitis, anti-α-amino-3-hydroxy-5-methyl-4-isoxazoleproprionic acid receptor (AMPAR) encephalitis, anti-gamma-aminobutyric acid receptor (GABAR) encephalitis, anti-contactin-associated protein-like 2 (Caspr2) encephalitis, anti-leucine-rich glioma inactivated protein 1 (LGI1) encephalitis, anti-dipeptidyl-peptidase-like protein 6 (DPPX) encephalitis, anti-glutamic acid decarboxylase (GAD) encephalitis, anti-glycine receptor encephalitis, etc.

Diagnosis of AE is challenging due to the similarity in clinical presentations of AE and other diseases (such as infectious encephalitis, inflammatory brain diseases, metabolic diseases and psychiatric disorders). Thus, autoantibody tests are extremely important for the proper diagnosis of AE. However, cell-based test for qualitatively and quantitatively detecting autoantibody is not yet available. In view of the foregoing, there exists in the related art a need of an improved method for qualitatively and quantitatively detecting autoantibody against neuronal protein for making diagnosis of AE.

### SUMMARY

As embodied and broadly described herein, the present disclosure is directed to a method of identifying and treating a subject having AE. The method includes:
(1) detecting an autoantibody against a neuronal antigen in a biological sample obtained from the subject comprising:
   (a) transfecting a host cell with a plasmid comprising a nucleic acid encoding the neuronal antigen to produce a transfected host cell having the neuronal antigen expressed thereon;
   (b) mixing the transfected host cell of step (a) and a control host cell having a first fluorophore labeled thereon to produce a mixture, in which the control host cell does not have the neuronal antigen thereon expressed thereon;
   (c) adding the biological sample to the mixture of step (b), in which the biological sample has been diluted at least 15 times before adding to the mixture of step (b);
   (d) adding an anti-human antibody to the product of step (c), wherein the anti-human antibody is conjugated to a second fluorophore.
   (e) irradiating the products of step (d) independently with a first light having a first wavelength to excite the first fluorophore and a second light having a second wavelength to excite the second fluorophore;
   (f) measuring respective signals emitted from the excited first and second fluorophores at a third and fourth wavelengths; and
   (g) subtracting the signal measured at the third wavelength from the signal measured at the fourth wavelength thereby producing a real signal, which indicates the presence of the autoantibody in the biological sample;
   wherein, both steps (b) and (c) are conducted in the same vessel; and
(2) treating the subject by administering solumedrol or immunoglobulin (Ig) to the subject to ameliorate symptoms associated with AE.

According to optional embodiments, in step (1), the method further comprises a washing and reacting step, in which after step (c) and before step (d), the product of step (c) is washed with a buffer comprising 1% bovine serum albumin (BSA), 2 mM ethylenediaminetetraacetic acid (EDTA), and 0.02% sodium azide (NaN₃).

According to preferred embodiments of the present disclosure, in step (c), the biological sample is diluted 20 times before adding to the mixture of step (b).

According to preferred embodiments of the present disclosure, both steps (c) and (d) are conducted at 4° C. for 20 minutes.

According to embodiments of the present disclosure, the first fluorophore is 2′-(4-ethoxyphenyl)-5-(4-methyl-1-piperazinyl)-2,5′-bi-1H-benzimidazole trihydrochloride, while the second fluorophore is fluorescein isothiocyanate (FITC).

According to embodiments of the present disclosure, in step (e), the products of step (d) is independently irradiated with the first light having the first wavelength of 340 nm, and the second light having the second wavelength of 490-495 nm; while in step (f), respective signals are measured at the third wavelength of 510 nm and the fourth wavelength of 510-530 nm.

Examples of the biological sample suitable for use in the present disclosure include, but are not limited to, whole blood, plasma, serum, cerebrospinal fluid, or biopsy samples from a human subject. In some embodiments, the biological sample is serum. In other embodiments, the biological sample is cerebrospinal fluid.

Examples of the host cell suitable for use in the present disclosure include, but are not limited to, human embryonic kidney (HEK) 293 cells, 293T Cells, Chinese Hamster Ovary (CHO) Cells, Baby Hamster Kidney-21 (BHK-21) Cells, Madin-Darby Canine Kidney (MDCK) Cells, 293-F cells, 293-H Cells and PER.C6 Cells. Optionally, the host cell is immobilized on a substrate.

Examples of the neuronal antigen suitable for use in the present disclosure include, but are not limited to, N-methyl-D-aspartate receptor (NMDAR), α-amino-3-hydroxy-5-methyl-4-isoxazoleproprionic acid receptor (AMPAR), gamma-aminobutyric acid receptor (GABAR), contactin-associated protein-like 2 (Caspr2), leucine-rich glioma inactivated protein 1 (LGI1), or dipeptidyl-peptidase-like protein 6 (DPPX).

According to some embodiments of the present disclosure, the plasmid comprises a nucleic acid encoding the NMDAR1 having a polynucleotide sequence of SEQ ID NO: 1.

According to some embodiments of the present disclosure, the plasmid comprises a nucleic acid encoding the GABAR having a polynucleotide sequence of SEQ ID NO: 2.

According to some embodiments of the present disclosure, the plasmid comprises a nucleic acid encoding the AMPAR having a polynucleotide sequence of SEQ ID NO: 3.

According to some embodiments of the present disclosure, the plasmid comprises a nucleic acid encoding the Caspr2 having a polynucleotide sequence of SEQ ID NO: 4. According to some embodiments of the present disclosure, the plasmid comprises a nucleic acid encoding the LGI1 having a polynucleotide sequence of SEQ ID NO: 5.

According to some embodiments of the present disclosure, the plasmid comprises a nucleic acid encoding the DPPX having a polynucleotide sequence of SEQ ID NO: 6.

Optionally or in addition, in step (1), the method further includes quantifying the autoantibody in the biological sample by:
(i) providing a control antibody specific to the neuronal antigen, wherein the control antibody is conjugated to the fluorophore;
(ii) serially diluting the control antibody to produce a plurality of diluted antibodies respectively having pre-determined concentrations;
(iii) respectively irradiating the plurality of diluted antibodies of step (ii) with the light having the first wavelength;
(iv) respectively measuring the intensities of signals emitted from each diluted antibodies of step (iii) at the second wavelength;
(v) constructing a calibration plot of the concentrations of the plurality of diluted antibodies as a function of the intensities of the signals measured in step (iv); and
(vi) quantifying the autoantibody from the calibration plot of step (v) based on the intensity of the real signal produced in step (g).

According to embodiments of the present disclosure, in step (2), the method further includes administering rituximab, cyclophosphamide or both to the subject.

Many of the attendant features and advantages of the present disclosure will become better understood with reference to the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1 depicts the non-specific interference signals originating from serum during the detection of autoantibody with antigen-expressing cells by (A) immunofluorescence staining assay, and (B) flow cytometry analysis in accordance with Example 1.2 of the present disclosure; and

FIG. 2 depicts the effects of serum dilution on the detection of autoantibodies with antigen-expressing cells by flow cytometry analysis in accordance with Example 1.3 of the present disclosure.

### DETAILED DESCRIPTION OF THE INVENTION

The detailed description provided below is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. DEFINITION

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “autoantibody” as used herein has its art understood meaning, and refers to an antibody that is produced by the immune system of a subject and is directed against one or more of the subject's own proteins. According to the embodiments of the present disclosure, the term “autoantibody” refers to an antibody that is directed to an endogenous protein of a subject, for example, NMDAR (e.g., NMDAR1), AMPAR, GABAR (e.g., GABAB2), Caspr2, LGI1, or DPPX. Autoantibodies may attack the subject's own cells, tissues, and/or organs thereby causing autoimmune diseases (e.g., AE).

The term “quantify, quantifying or quantification” as used herein refers to a process of determining the quantity or concentration of a substance (e.g., an autoantibody) in a sample, whether relative or absolute. For example, quantification may be determined by methods including but not limited to, flow cytometry, enzyme-linked immunosorbent assay (ELISA), spectrophotometry, micro-array, and indirect immunofluorescent test (IFT). The quantity or concentration may be expressed in any unit of measure. According to some embodiments of the present disclosure, the concentration of the substance (e.g., the autoantibody) in the sample is expressed as microgram per milliliter (μg/mL).

As used herein, the term “detect, detecting or “detection”, in addition to art-recognized meanings, refers to any process of observing an autoantibody, in a biological sample, whether the autoantibody is observed. In other words, the act of assaying a sample for the determination of the presence or absence of an autoantibody is a “detection” even if the autoantibody is eventually found not present in the sample or present at a low level beyond the sensitivity of the detecting method. Detection may be a quantitative, semi-quantitative or non-quantitative observation.

The term “polynucleotide” and “nucleic acid” are used interchangeably in the present disclosure and is understood to mean, according to the present disclosure, either a double-stranded DNA, a single-stranded DNA or a product of transcription of said DNA (e.g., RNA molecule). It should also be understood that the present disclosure does not relate to genomic polynucleotides in their natural environment or natural state. The polynucleotide of the present invention can be isolated, purified (or partially purified), by separation methods including, but not limited to, ion-exchange chromatography, molecular size exclusion chromatography, or by genetic engineering methods such as amplification, subtractive hybridization, cloning, sub-cloning or chemical synthesis, or combinations of these genetic engineering methods.

The term “sequence identity” as used herein refers to the sequence relationships between two or more nucleic acids when aligned for maximum correspondence over a specified comparison window. The percentage of “identity” is determined by comparing two optimally aligned sequences over the comparison window. For “optimal alignment” of the two sequences, it will be appreciated that the portion of the sequence in the comparison window may include gaps (e.g., deletions or additions) as compared to the reference sequence, which does not contain additions or deletions. After alignment, the number of matched positions (i.e., positions where the identical nucleic acid base occurs in both sequences) is determined and then divided by the total number of positions in the comparison window. This result is then multiplied by 100 to calculate the percentage of sequence identity. In some embodiments, two sequences have the same total number of nucleotides. The aligned sequences can be analyzed by any method familiar with one skilled artisan, including GAP, BESTFIT, BLAST, FASTA, and TFASTA.

As used herein, the term “transfect or transfecting” refers to the introduction of a polynucleotide into a cell via use of a plasmid or vector. The nucleic acid of the polynucleotide may be in the form of naked DNA or RNA, associated with various proteins. The term “transfect or transfecting” as used herein is intended to convey the broadest possible meaning and encompass the introduction, for example by transfection method (introducing a polynucleotide into eukaryotic cells by physical and/or chemical treatment), transformation method (introducing a polynucleotide into prokaryotic cells by physical and/or chemical treatment), viral method/viral transduction method (introducing a polynucleotide into eukaryotic and/or prokaryotic cells by a virus or a viral vector), conjugation method (introducing a polynucleotide from one cell to another cell by direct cell-to-cell contact or by a cytoplasmic bridge between the cells), and fusion method (fusing two cells, including homotypic cell fusion and heterotypic cell fusion).

The term “diagnosis” or “diagnosing” as used herein refers to methods by which a skilled artisan can estimate and/or determine the probability (“a likelihood”) of whether or not a patient is suffering from a given disease or condition (e.g., AE). In the case of the present invention, “diagnosis” includes using the detection results of the present invention, optionally together with other clinical characteristics, to arrive at a diagnosis (that is, the occurrence or nonoccurrence) of AE for the subject from which a sample was obtained and assayed. That such a diagnosis is “determined” is not meant to imply that the diagnosis is 100% accurate. Different biomarkers are indicative of multiple conditions. A skilled clinician does not use a single biomarker result in an informational vacuum, but rather the test result is used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

The term “fluorophore” used herein means a molecule which absorbs energy of a specific wavelength (i.e., a first wavelength) and re-emit energy at a different wavelength (i.e., a second wavelength).

The term “subject” is intended to refer to both the male and female gender unless one gender is specifically indicated.

### II. Methods for Detecting Autoantibodies Against a Neuronal Antigen

The present disclosure aims at providing a method of detecting an autoantibody against a neuronal antigen in a human biological sample. The method comprises,
(a) transfecting a host cell with a plasmid comprising a nucleic acid encoding the neuronal antigen to produce a transfected host cell having the neuronal antigen expressed thereon;
(b) mixing the transfected host cell of step (a) and a control host cell to produce a mixture, in which the control host cell is labeled with a first fluorophore and does not have the neuronal antigen expressed thereon;
(c) adding the biological sample to the mixture of step (b), in which the biological sample has been diluted at least 15 times before adding to the mixture of step (b);
(d) adding an anti-human antibody to the product of step (c), wherein the anti-human antibody is conjugated to a second fluorophore.
(e) irradiating the products of step (d) independently with a light having a first wavelength to excite the first fluorophore and a light having a second wavelength to excite the second fluorophore;
(f) measuring respective signals emitted from the excited first and second fluorophores at third and fourth wavelengths; and
(g) subtracting the signal measured at the third wavelength from the signal measured at the fourth wavelength thereby producing a real signal, which indicates the presence of the autoantibody in the biological sample;
wherein, steps (b) and (c) are conducted in the same vessel.

Depending on intended use, the autoantibody detected by the present method may be an autoantibody against NMDAR (i.e., an anti-NMDAR antibody that targets NMDAR and relates to anti-NMDAR encephalitis), an autoantibody against AMPAR (i.e., an anti-AMPAR antibody targets AMPAR and relates to anti-AMPAR encephalitis), an autoantibody against GABAR (i.e., an anti-GABAR antibody that targets GABAR and relates to anti-GABAR encephalitis), an autoantibody against Caspr2 (i.e., an anti-Caspr2 antibody that targets Caspr2 and relates to anti-Caspr2 encephalitis), an autoantibody against LGI1 (i.e., an anti-LGI1 antibody that targets LGI1 and relates to anti-LGI1 encephalitis), or an autoantibody against DPPX (i.e., an anti-DPPX antibody that targets DPPX and relates to anti-DPPX encephalitis). According to one preferred embodiment, the autoantibody is an anti-NMDAR1 antibody, which recognizes and targets the NR1 subunit of the NMDAR (NMDAR1). According to another preferred embodiment, the autoantibody is an anti-GABAB2 antibody, which recognizes and targets GABA B receptor 2 (GABAB2).

According to various embodiments of the present disclosure, the biological sample may be a sample of whole blood, plasma, serum, cerebrospinal fluid (CSF), or biopsy taken from a live subject. In certain preferred embodiments, the biological sample is serum taken from a human subject. In certain preferred embodiments, the biological sample is CSF taken from a human subject.

In step (a), a plasmid comprising a nucleic acid encoding the desired neural antigen is constructed and transfected into a host cell at conditions that allows the neuronal antigen to be expressed on the surface of the host cell. Depending on intended use, the host cell may be a prokaryotic cell or a eukaryotic cell. A skilled artisan in the related field may choose suitable cell as the host cell according to the protein (i.e., the neural antigen) intended to be expressed. Preferably, the host cell is a mammalian cell. In certain preferred embodiments, the host cell is HEK293 cell.

In one exemplary embodiment, the plasmid comprises a nucleic acid encoding the NMDAR1 having a polynucleotide sequence of SEQ ID NO: 1. It is known in the art that the codons encoding one amino acid residue may differ in any of their three positions (a phenomenon known as “codon degeneracy” or “codon redundancy”); for example, the glutamic acid (E) residue may be encoded by “GAA” or “GAG” codon (difference in the third position); the leucine (L) residue may be encoded by “TTA”, “TTG”, “CTT”, “CTC”, “CTA” or “CTG” codon (difference in the first or third position); and serine(S) residue may be encoded by “TCA”, “TCG”, “TCC”, “TCT”, “AGT” or “AGC” codon (difference in the first, second, or third position). Based on codon degeneracy, a skilled artisan may substitute one or more nucleotides of the polynucleotide without affecting the amino acid sequence encoded therefrom. Accordingly, the polynucleotide comprising one or more degenerate codons are intended to be included within the scope of the present disclosure. According to various embodiments, the nucleic acid encoding the NMDAR1 having a polynucleotide sequence at least 85% (e.g., 85%, 86%, 87%, 88%, 89%, 90%, 91%, 92%, 93%, 94%, 95%, 96%, 97%, 98%, 99% or 100%) identical to SEQ ID NO: 1; preferably, at least 90% identical to SEQ ID NO: 1; more preferably, at least 95% identical to SEQ ID NO: 1.

In one exemplary embodiment, the plasmid comprises a nucleic acid encoding the GABAB2 having a polynucleotide sequence of SEQ ID NO: 2. As described above, a skilled artisan may substitute one or more nucleotides of the polynucleotide without affecting the amino acid sequence encoded therefrom. According to various embodiments, the nucleic acid encoding the GABAB2 comprises the polynucleotide sequence at least 85% identical to SEQ ID NO: 2; preferably, at least 90% identical to SEQ ID NO: 2; more preferably, at least 95% identical to SEQ ID NO: 2.

In one exemplary embodiment, the plasmid comprises a nucleic acid encoding the AMPAR having a polynucleotide sequence of SEQ ID NO: 3. Based on codon degeneracy, a skilled artisan may substitute one or more nucleotides of the polynucleotide without affecting the amino acid sequence encoded therefrom. According to various embodiments, the nucleic acid encoding the AMPAR comprises the polynucleotide sequence at least 85% identical to SEQ ID NO: 3; preferably, at least 90% identical to SEQ ID NO: 3; more preferably, at least 95% identical to SEQ ID NO: 3.

In another exemplary embodiment, the plasmid comprises a nucleic acid encoding the Caspr2 having a polynucleotide sequence of SEQ ID NO: 4. Based on codon degeneracy, one or more nucleotides of the polynucleotide may be substituted. According to various embodiments, the nucleic acid encoding the Caspr2 comprises the polynucleotide sequence at least 85% identical to SEQ ID NO: 4; preferably, at least 90% identical to SEQ ID NO: 4; more preferably, at least 95% identical to SEQ ID NO: 4.

In an alternative embodiment, the plasmid comprises a nucleic acid encoding the LGI1 having a polynucleotide sequence of SEQ ID NO: 5. Based on codon degeneracy, the nucleic acid encoding the LGI1 may comprise one or more degenerate codons. According to various embodiments, the nucleic acid encoding the LGI1 comprises the polynucleotide sequence at least 85% identical to SEQ ID NO: 5; preferably, at least 90% identical to SEQ ID NO: 5; more preferably, at least 95% identical to SEQ ID NO: 5.

In an alternative embodiment, the plasmid comprises a nucleic acid encoding the DPPX having a polynucleotide sequence of SEQ ID NO: 6. Based on codon degeneracy, the nucleic acid encoding the DPPX may comprise one or more degenerate codons. According to various embodiments, the nucleic acid encoding the DPPX comprises the polynucleotide sequence at least 85% identical to SEQ ID NO: 6; preferably, at least 90% identical to SEQ ID NO: 6; more preferably, at least 95% identical to SEQ ID NO: 6.

Non-limiting examples of methods for transfecting the plasmid into a cell include, but are not limited to, calcium phosphate co-precipitation, electroporation, nucleofection, cell squeezing (gently squeezing the cell membrane), sonoporation (inducing pore formation in cell membrane by high-intensity ultrasound), optical transfection (generating a tiny hole in cell membrane by highly focused laser), impalefection (delivering polynucleotides by nanomaterials, such as nanofibers, nanotubes or nanowires), gene gun (delivering polynucleotide-coated nanoparticles using a biolistic device or gene gun), magnetofection (delivering polynucleotide using magnetic force), viral transduction (using viruses as a carrier to deliver polynucleotides into target cells), or transfection via a dendrimer, a liposome, or a cationic polymer. According to some preferred embodiments, the plasmid is transfected into the host cell via a liposome. Specifically, the plasmid is mixed with lipid subunits that form liposomes in an aqueous environment, followed by adding the mixture to the host cell, and incubated at an appropriate temperature (e.g., 37° C.) for a period (e.g., 24 hours) allowing the plasmid entering into the host cell for expression.

Conventionally, for detection purpose, after step (a), the biological sample is divided into two parts and respectively mixed with the transfected host cell and a control host cell in separate containers (e.g., tubes). In the present disclosure, however, instead of operating in separate tubes as taught in conventional method, a One-Tube strategy is adopted to eliminate sampling errors caused by operations in separate containers. Specifically, the transfected host cell having the target neuronal antigen expressed thereon is mixed with a control host cell to produce a first mixture, in which the host cell is labeled with a first fluorophore (e.g., Hoechst 33342) and does not have a neuronal antigen expressed thereon (step (b)); then, a biological sample is added to the first mixture of step (b) (step (c)). Note that both steps (b) and (c) are conducted in the same tube. This one-tube strategy ensures that both cell types are subjected to identical reaction conditions, thereby avoiding pipetting errors arising from separate sampling steps, which in turn significantly enhances quantitative accuracy, and prevents false negatives or numerical drifts that may occur after background subtraction. According to embodiments of the present disclosure, in step (c), the biological sample is preferably diluted at least 15 times (e.g., 15, 16, 17, 18, 19, or 20 times); preferably, at least 20 times, prior to being added to the mixture of step (b). According to embodiments, the dilution ensures that the signal peaks between the antigen-transfected cell and the control host cell can be best differentiated, thereby achieving an optimal signal-to-noise ratio and successfully overcoming the nuclear-like background interference specific to serum. In step (c), when the autoantibody is present in the biological sample, it would be captured by the neuronal antigen (e.g., NMDAR, AMPAR, GABAR, Caspr2, LGI1, or DPPX) expressed on the surface of the host cell, forming an antigen-antibody immunocomplex. By contrast, when the autoantibody is absent from the biological sample, no immunocomplex forms after incubation. According to preferred embodiments of the present disclosure, both step (b) and step (c) are conducted at 4° C. for 20 minutes.

Optionally or in addition, after step (c) and prior to step (d), the method may further include a washing step, in which the product of step (c) is washed and reacted in an optimized buffer to remove unbound autoantibodies. Specifically, the optimized buffer is a phosphate-buffered saline (PBS) comprising 1% bovine serum albumin (BSA), 2 mM ethylenediaminetetraacetic acid (EDTA), and 0.02% sodium azide (NaN₃). According to embodiments of the present disclosure, the adoption of the optimized buffer successfully removes serum components (e.g., fetal bovine serum (FBS)) and maintains cell morphology thereby further reduces background signal interference.

In step (d), an anti-human antibody is added to the product of step (c). The mixture is preferably incubated at an appropriate temperature (e.g., 18-25° C.) for a sufficient period of time (e.g., 30 minutes). According to certain embodiments, the anti-human antibody is conjugated to a second fluorophore. In this way, when the antigen-antibody immunocomplex is formed in the product of step (c), then the anti-human antibody would recognize and bind to the autoantibody (e.g., the fragment crystallizable region (Fc region) of the autoantibody) of the antigen-antibody immunocomplex. The thus-formed antigen-antibody-antibody immunocomplex may then be detected via measuring the signal emitted by the second fluorophore. By contrast, no antigen-antibody-antibody immunocomplex would be formed in step (d) in the case when the autoantibody is absent in the biological sample and no immunocomplex is thus-formed in the product of step (c).

Examples of the first fluorophore suitable to use in the present method include, but are not limited to, SYTO dyes, LUCS®9, 2′-(4-ethoxyphenyl)-5-(4-methyl-1-piperazinyl)-2,5′-bi-1H-benzimidazole trihydrochloride (Hoechst 33342) and the like. According to one exemplary embodiment, the first fluorophore is Hoechst 33342. Examples of the second fluorophore suitable to use in the present method include, but are not limited to, cyan fluorescent protein (CFP), green fluorescent protein (GFP), yellow fluorescent protein (CFP), red fluorescent protein (CFP), far-red fluorescent protein (FFP), infrared fluorescent protein (IFP), fluorescein isothiocyanate (FITC), phycoerythrin (PE), allophycocyanin (APC), carboxy-X-rhodamine (ROX), Texas red, cyanine dye (for example, Cy2, Cy3, Cy5 or Cy7), and Alexa Fluor® dye (such as, Alexa Fluor®488, Alexa Fluor®430, Alexa Fluor®532, Alexa Fluor®546, Alexa Fluor®555, Alexa Fluor®568, Alexa Fluor®594, Alexa Fluor®644, Alexa Fluor®660 or Alexa Fluor®680). According to one exemplary embodiment, the second fluorophore is FITC.

It would be appreciated that instead of the fluorophore, the anti-human antibody may alternatively being conjugated with other reporter molecules, e.g., a quantum dot, as long as such reporter molecules are countable by suitable assays.

Next, the product of step (d) is independently irradiated with a first light having a first wavelength to excite the first fluorophore and a second light having a second wavelength to excite the second fluorophore (step (e)), and the intensities of respective signals emitted by the excited first and second fluorophores are then measured at third and fourth wavelengths (step (f)). The signal intensity may be determined by any method discussed above, for example, flow cytometry. As could be appreciated, the wavelength of the light vary with the type of the fluorophore, and a skilled artisan would be able to choose suitable wavelengths to excite the first or second fluorophores and measure signal intensity emitted therefrom. For example, in certain embodiments, the first fluorophore is Hoechst 33342 and the second fluorophore is FITC; accordingly, a light of 340 nm may be used to excite Hoechst 33342 to give an emission signal at the wavelength of about 510 nm; while a light of 485-495 nm may be used to excite FITC to give an emission signal at the wavelength ranges from 510 nm to 530 nm.

In step (g), the signal measured at the third wavelength is subtracted from the signal measured at the fourth wavelength thereby producing a real signal, which indicates the presence of the autoantibody in the biological sample.

According to embodiments of the present disclosure, the concentration of the autoantibody in the biological sample may be calculated via use of a calibration plot, which is established by a method comprising steps of:
(i) providing a control antibody specific to the neuronal antigen, wherein the control antibody is conjugated to the second fluorophore;
(ii) serially diluting the control antibody to produce a plurality of diluted antibodies respectively having pre-determined concentrations;
(iii) respectively irradiating the plurality of diluted antibodies of step (ii) with the light having the second wavelength;
(iv) respectively measuring the intensities of signals emitted from each diluted antibodies of step (iii) at the fourth wavelength;
(v) constructing a calibration plot of the concentrations of the plurality of diluted antibodies as a function of the intensities of the signals measured in step (iv); and
(vi) quantifying the autoantibody from the calibration plot of step (v) based on the intensity of the real signal produced in step (g).

The control antibody specific to the neuronal antigen may be any one of an anti-NMDAR antibody, an anti-AMPAR antibody, an anti-GABAR antibody, an anti-Caspr2 antibody, an anti-LGI1 antibody, or an anti-DPPX antibody. Further, the control antibody is conjugated to a third fluorophore, which is same as the second fluorophore conjugated to the anti-human antibody described above. According to embodiments of the present disclosure, both the second and third fluorophores (i.e., the fluorophore conjugated to the anti-human antibody, and the fluorophore conjugated to the control antibody) are FITC. According to certain embodiments, both the second and third fluorophores are PE.

The fluorophore-conjugated control antibody is serially diluted to produce a plurality of diluted antibodies respectively having pre-determined concentrations; for example, a plurality of diluted antibodies respectively having the concentrations of 10 μg/ml, 5 g/ml, 2.5 μg/ml, 1.25 μg/ml, 0.625 μg/ml, 0.3125 μg/ml, and 0.156 μg/ml. The diluted antibodies are then irradiated with the light having the second wavelength to excite the second fluorophores of the diluted antibodies, and the intensities of the signals emitted by the second fluorophores are measured at the fourth wavelength. For example, as described above, the FITC may be excited at the wavelength of 485-495 nm, and the signal/signal intensity emitted therefrom may be measured at the wavelength of 510-530 nm. The concentration of diluted antibodies would be proportional to the intensity of signal emitted therefrom. A calibration plot may thus be constructed, in which the x-coordinate corresponds to the concentration of diluted antibodies, and the y-coordinate corresponds to the average fluorescence intensity emitted therefrom. The concentration of autoantibody in the biological sample is accordingly determined from the calibration plot based on the fluorescence intensity thereof (i.e., the real signal of step (g)).

### III. Methods for Diagnosing and Treating a Subject Suspected of Having AE

The autoantibody concentration thus obtained by the method described above is useful in rendering a diagnosis of AE in the subject. Specifically, in the case when the concentration of the autoantibody in the biological sample derived/isolated from a subject is higher than that of the autoantibody in a control sample derived/isolated from a healthy subject or a reference sample (for example, the reference data obtained from gene/protein expression databases that are normalized to allow direct quantitative comparison with the data from the test sample), then the subject is determined to have AE. By contrast, in the case when the concentration of the autoantibody in the biological sample derived/isolated from a subject equal to or is lower than that of the autoantibody in a control sample derived/isolated from a healthy subject or a reference sample, then the subject is determined to be not having AE. Based on the diagnostic results, a suitable treatment (such as corticosteroids, intravenous immunoglobulins (IVIG), plasmapheresis, rituximab, cyclophosphamide, etc.) may be administered to the subject in need thereof in time.

According to one exemplary embodiment, the method is used to diagnose anti-NMDAR1 encephalitis (i.e., the encephalitis caused by anti-NMDAR1 autoantibody). In the embodiment, the concentration of the anti-NMDAR1 autoantibody higher than 0.73 μg/mL indicates that the subject has anti-NMDAR1 encephalitis.

Once the subject is determined to have AE, suitable treatment may be administered to the subject to ameliorate or alleviate symptoms associated with AE. First-line therapy for AE may be intravenous injection (IV) of solumedrol at the dose of 1 gram daily for 3-5 days then a taper over several weeks; or IV of immunoglobulin (Ig) at the dose of 0.4 g/kg/day for 5 days. Optionally or in addition, second-line treatment may be used if first-line therapy fails. Second line therapies include rituximab (often 375 mg/m2 weekly for 4 weeks) or cyclophosphamide (750 mg/m2 IV monthly until improvement is noted), or both.

### IV. Kits for Detecting Autoantibody and Uses Thereof

The present disclosure thus also pertains to kits for detecting autoantibodies and uses thereof in determining a cause of AE.

According to some embodiments of the present disclosure, the kit comprises one or more plasmids, and an anti-human antibody, in which each plasmid encodes a polynucleotide having a polynucleotide sequence at least 85% identical to SEQ ID NOs: 1, 2, 3, 4, 5, or 6; preferably, at least 90% identical to SEQ ID NOs: 1, 2, 3, 4, 5, or 6; more preferably, at least 95% identical to SEQ ID NOs: 1, 2, 3, 4, 5, or 6. In one specific example, each polynucleotide has the polynucleotide sequence of SEQ ID NOs: 1, 2, 3, 4, 5, or 6 (i.e., the nucleotide sequence 100% identical to SEQ ID NOs: 1, 2, 3, 4, 5, or 6). According to the embodiments, the anti-human antibody has a fluorophore conjugated thereto.

According to some preferred embodiments, the present kit comprises a first to a sixth plasmids respectively designated as “NMDAR 1plasmid”, “GABAB2 plasmid”, “AMPAR plasmid”, “Caspr2 plasmid”, “LGI1 plasmid” and “DPPX plasmid”. In the embodiments, the NMDAR1 plasmid comprises a nucleic acid encoding the NMDAR of SEQ ID NO: 1; the GABAB2 plasmid comprises a nucleic acid encoding the GABAB2 of SEQ ID NO: 2; the AMPAR plasmid comprises a nucleic acid encoding the AMPAR of SEQ ID NO: 3; the Caspr2 plasmid comprises a nucleic acid encoding the Caspr2 of SEQ ID NO:4; the LGI1 plasmid comprising a nucleic acid encoding the LGI1 of SEQ ID NO:5; and the DPPX plasmid comprising a nucleic acid encoding the DPPX of SEQ ID NO:6.

According to some exemplary embodiments, any of the polynucleotide described above present in the plasmid is operably linked to one or more regulatory sequences (e.g., promoter and enhancer) that control the expression of the polynucleotide in the host cell. Depending on the intended applications, the promoter may be an inducible promoter or a constitutive promoter. As known in the art, an inducible promoter refers to refers to a promoter that selectively expresses a coding sequence or functional RNA in response to the presence of an endogenous or exogenous stimulus, e.g., chemical compound, environmental stimulus, hormonal stimulus, or developmental signaling. By contrast, a constitutive promoter refers to a promoter whose activity is maintained at a relatively constant level in all cells of an organism with little or no regard to cell environmental conditions (as the concentration of a substrate). Examples of the inducible promoter suitable for use in the present disclosure include, but are not limited to, heat shock inducible promoter, metallothionin promoter, ecdysone-inducible promoter, FKBP dimerization inducible promoter, Gal4-estrogen receptor fusion protein regulated promoter, steroid inducible promoter, streptogramin responsive promoter, and tetracycline regulated promoter. Exemplary constitutive promoters suitable for use in the present disclosure include, but are not limited to, cytomegalovirus (CMV) promoter, rous sarcoma virus (RSV) promoter, simian virus (SV40) promoter, mouse mammary tumor virus (MMTV) promoter, phosphoglycerate kinase (PGK) promoter, chicken beta-active promoter, elongation factor 1-alpha (EF1-a) promoter, human H1 promoter, and U6 promoter. According to one exemplary embodiment, the promoter is CMV promoter.

Also disclosed herein is a method of identifying a source of AE in a human subject via a biological sample isolated from the human subject by using the present kit. The method comprises,
(a) respectively introducing the NMDAR1, GABAB2, AMPAR, Caspr2, LGI1 and DPPX plasmids into a first to a sixth populations of a host cell (e.g., HEK 293 cell);
(b) respectively mixing the first to sixth cell populations of step (a) with a control host cell to produce a first to sixth mixtures, in which the control host cell is labeled with a first fluorophore and does not have any one of NMDAR1, GABAB2, AMPAR, Caspr2, LGI1, or DPPX antigen expressed thereon;
(c) respectively adding the biological sample to the first to sixth mixtures of step (b) to produce a first to a sixth products, in which the biological sample is diluted at least 15 times before being added to each of the first to sixth mixtures of step (b);
(d) adding the anti-human antibody to the first to sixth products of step (c) to produce first to sixth antibody mixtures, wherein the anti-human antibody is conjugated to a second fluorophore;
(e) respectively irradiating the first to sixth antibody mixtures of step (d) with a light having a first and second wavelengths;
(f) respectively measuring the signals emitted from the first to sixth antibody mixtures at a third and fourth wavelengths; and
(g) subtracting the signal measured at the third wavelength from the signal measured at the fourth wavelength thereby producing first to sixth real signals respective for the first to sixth antibody mixtures, in which each of the first to sixth real signals indicates the presence of the autoantibody in the biological sample.

The steps (a)-(f) of the method are quite similar to those of the detection method described in section (ii) of the present disclosure. Hence, the detailed description is omitted herein for the sake of brevity.

In step (g), a skilled artisan may identify the cause of AE based on the real signal produced in step (g). Specifically, the first to sixth cell populations respectively express the NMDAR1, GABA_{B2}, AMPAR, Caspr2, LGI1 and DPPX on their surfaces. Since the first to sixth antibody mixtures respectively correspond to the first to sixth cell populations, the detection of the fluorescence signal in one specific antibody mixture (e.g., the first antibody mixture) indicates the antigen-antibody-antibody immunocomplex is formed on surface of the corresponding cell population (e.g., the immunocomplex comprising the NMDAR1, anti-NMDAR1 autoantibody and anti-human antibody is formed on the surface of the first cell population); by this way, the corresponding autoantibody (e.g., the NMDAR1) present in the biological sample is identified, and the cause of AE (e.g., anti-NMDAR1 encephalitis) may thus be determined.

According to one embodiment, the fluorescence signal detected in the first antibody mixture indicates that the anti-NMDAR1 autoantibody is present in the biological sample, and the AE may thus be identified as being caused by or related to the anti-NMDAR1 autoantibody (i.e., being an anti-NMDAR1 AE). According to one embodiment, the fluorescence signal detected in the second antibody mixture indicates that the anti-GABAB2 autoantibody is present in the biological sample, and the AE may thus be identified as being caused by or relates to the anti-GABAB2 autoantibody (i.e., being an anti-GABAB2 AE). According to another embodiment, the fluorescence signal detected in the third antibody mixture indicates that the anti-AMPAR autoantibody is present in the biological sample, and the AE may thus be identified as being caused by or relates to the anti-AMPAR autoantibody (i.e., being an anti-AMPAR AE). According to another embodiment, the fluorescence signal detected in the fourth antibody mixture indicates that the anti-Caspr2 autoantibody is present in the biological sample, and the AE may thus be identified as being caused by or relates to the anti-Caspr2 autoantibody (i.e., being an anti-Caspr2 AE). According to still another embodiment, the fluorescence signal detected in the fifth antibody mixture indicates that the anti-LGI1 autoantibody is present in the biological sample, and the AE may thus be identified as being caused by or relates to the anti-LGI1 autoantibody (i.e., being an anti-LGI1 AE). According to a further embodiment, the fluorescence signal detected in the sixth antibody mixture indicates that the anti-DPPX autoantibody is present in the biological sample, and the AE may thus be identified as being caused by or relates to the anti-DPPX autoantibody (i.e., being an anti-DPPX AE).

According to various embodiments, the biological sample may be a sample of whole blood, plasma, serum, cerebrospinal fluid, or biopsy taken from a human subject. In some preferred embodiments, the biological sample is serum of a human subject.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Plasmid Construction

Six plasmids were constructed to respectively comprise nucleic acid (i.e., any one of SEQ ID NOs: 1 to 6) encoding the NMDAR1, GABAB2, AMPAR, Caspr2, LGI1 and DPPX polypeptides. The details and operating conditions of plasmids are provided in Table 1.

TABLE 1
Detail description of the present plasmids

| | | ORF seq. | | | | | |
|---|---|---|---|---|---|---|---|
| | | (SEQ ID | | | | E. coli | Mammalian |
| Plasmid | Vector | NO) | ORF Ref seq. | ORF size | Tagged | selection | selection |
| NMDAR1 | pCMV6- | 1 | NM_007327 | 2814 | Myc-DDK | Kanamycin | Neomycin |
| | Entry | | | | (C) | | |
| GABA_{B2} | pCMV6- | 2 | NM_021904 | 2697 | Myc-DDK | Kanamycin | Neomycin |
| | Entry | | | | (C) | | |
| AMPA | pCMV6- | 3 | NM_000827 | 2718 | Myc-DDK | Kanamycin | Neomycin |
| | Entry | | | | (C) | | |
| Caspr2 | pCMV6- | 4 | NM_014141 | 3993 | Myc-DDK | Kanamycin | Neomycin |
| | Entry | | | | (C) | | |
| LGI1 | pCMV6- | 5 | NM_005097 | 1671 | Myc-DDK | Kanamycin | Neomycin |
| | Entry | | | | (C) | | |
| DPPX | pCMV6- | 6 | NM_001039350 | 2403 | Myc-DDK | Kanamycin | Neomycin |
| | Entry | | | | (C) | | |

### Cell Transformation

HEK293 cells (5×10⁵ cells/well) were seeded in 6-well plates. Twenty four hours later, the HEK293 cells were transformed by DNA plasmids with the aid of LIPOFECTAMINE™ 2000 in accordance with the manufacturer's instructions. Briefly, the plasmid (3 μg) was diluted in 50 μL of OPTI-MEM™ I medium. After diluting LIPOFECTAMINE™ 2000 (6 μL) in 50 μL of OPTI-MEM™ I medium and incubating at room temperature for 5 minutes, the diluted plasmid was mixed with the diluted LIPOFECTAMINE™ 2000. The mixture was incubated at room temperature for 20 minutes, and then added to the HEK293 cells. The medium was replaced with fresh culture medium (DMEM containing 10% fetal bovine serum) six hours post-transfection. The cells were incubated at 37° C. in a CO₂ incubator for 24 hours prior to the expression and antibody-binding tests.

### Flow Cytometry

Plasmid-transfected cells (1×10⁵ cells) were fixed with 80% acetone or 4% paraformaldehyde (PFA) and blocked with blocking buffer, followed by incubating at room temperature for 1 hour. The plasmid-transfected cells were first mixed with a control to produce a first mixture, note that the control were HEK293 cells pre-labeled with Hoechst 33342 dye and did not have any neuronal antigen expressed thereon. Serum samples derived from AE patients were diluted various times (e.g., 10, 15 or 20 times) in PBS containing 10% fetal bovine serum (FBS) and then added to the first mixture to produce a second mixture. The second mixture was incubated at 4° C. for 20 minutes. An anti-NMDAR, anti-GABAR, anti-AMPAR, anti-Caspr2, anti-LGI1 or anti-DPPX antibody (each derived from human or mouse source) served as a positive control antibody in the study. After washing the cells with an optimized buffer (PBS containing 1% BSA, 2 mM EDTA and 0.02% NaN₃) for 3 times by centrifugation at 1,500 rpm for 5 minutes, FITC-conjugated anti-mouse antibody or FITC-conjugated anti-human antibody (each serving as the secondary antibody) was added to the cells and incubated at room temperature for 30 minutes. The cells were washed with the optimized buffer for 3 times by centrifugation at 1,500 rpm for 5 minutes. The signal intensity of the cells were determined by flow cytometry analysis with excitation wavelengths of 340 nm and 488 nm and emission wavelengths of 510 nm and 530 nm. Indirect immunofluorescent test (IFT)

Plasmid-transfected cells were seeded on glass slides pre-coated with poly-L-lysine, followed by fixing with 80% acetone or 1% PFA. Blocking buffer, 1% NaN₃ in PBS containing 10% FBS, was added to the fixed cells. After incubating at room temperature for 1 hour, serum samples derived from AE patients (diluted in PBS containing 0.1% TWEEN® 20 (PBS-T)) were respectively added to the plasmid-transfected cells and incubated at room temperature for 30 minutes. Then, the cells were washed with PBS-T for 5 times, and then, FITC-conjugated anti-human antibody or anti-mouse antibody (serving as the secondary antibody) was added to the cells and incubated at room temperature for 30 minutes. The cells were washed with PBS-T for 5 times. The signal was detected by fluorescence microscope. An anti-NMDAR, anti-GABAR, anti-AMPAR, anti-Caspr2, anti-LGI1 or anti-DPPX antibody (each derived from human or mouse) served as a positive control antibody.

### Example 1: Establishing the Present Method for the Detection of Autoantibodies Against Neuronal Antigens with Flow Cytometry

### 1.1 Detection of Autoantibodies in Serum by Antigen-Expressing Cells

Whether the plasmid-transfected cells may serve as a platform to detect antibody against specific neuronal antigen via flow cytometry was examined in the example. As described in “Materials and Methods” section, HEK293 cells were respectively transfected with six different plasmids to produce six populations of transfected HEK293 cells respectively expressing neuronal antigens (including NMDAR, GABAR, AMPAR, Caspr2, LGI1 and DPPX polypeptides) thereon, then the positive control antibody or serum samples of AE patients were added therein. The signal intensity of the cells was measured by flow cytometry.

The results indicated that each of the neuronal antigen-expressing cells (including the NMDAR-, GABAR-, AMPAR-, Caspr2-, LGI1- and DPPX-expressing cells) could detect its corresponding positive control antibody (i.e., the anti-NMDAR, anti-GABAR, anti-AMPAR, anti-Caspr2, anti-LGI1 or anti-DPPX antibody) (data not shown). Of note, the signal intensity of the NMDAR-expressing cells fixed by 80% acetone was higher than that of the NMDAR-expressing cells fixed by 4% PFA (data not shown). Also, compared to the cells fixed by 4% PFA, the signal-to-noise ratio (S/N or SNR) was greatly improved in the NMDAR-expressing cells fixed by 80% acetone (data not shown). The data further demonstrated that the fluorescence signal could be clearly detected in the GABAR- and AMPAR-expressing cells fixed by 4% PFA (data not shown).

### 1.2 Serum Significantly Affected the Detection of Autoantibodies

Next, the plasmid-transfected cells as described in Example 1.1 were used to detect autoantibodies present in serum samples derived from AE patients. To quantify the autoantibody against neuronal antigen, a calibration plot was first established using a positive control antibody. Specifically, the positive control antibody was serially diluted to produce a plurality of diluted antibodies respectively having concentrations of 10, 5, 2.5, 1.25, 0.625, 0.3125, and 0.156 μg/ml. After incubating with the neuronal antigen-expressing cells and mixing with FITC-conjugated secondary antibody, the signal intensities of each mixture were determined by flow cytometry. A calibration plot was thus constructed, where the x-axis represented the concentration of the diluted antibody, and the y-axis represented the signal intensity (geometric mean fluorescence intensity, geoMFI) of each diluted antibody. Then, the serum sample of AE patient was added to the neuronal antigen-expressing cells and mixed with FITC-conjugated secondary antibody and analyzed by flow cytometry as described in the “Materials and Methods” section. The concentration of the autoantibody against neuronal antigen in the serum sample was determined from the calibration plot based on the signal intensity measured by flow cytometry.

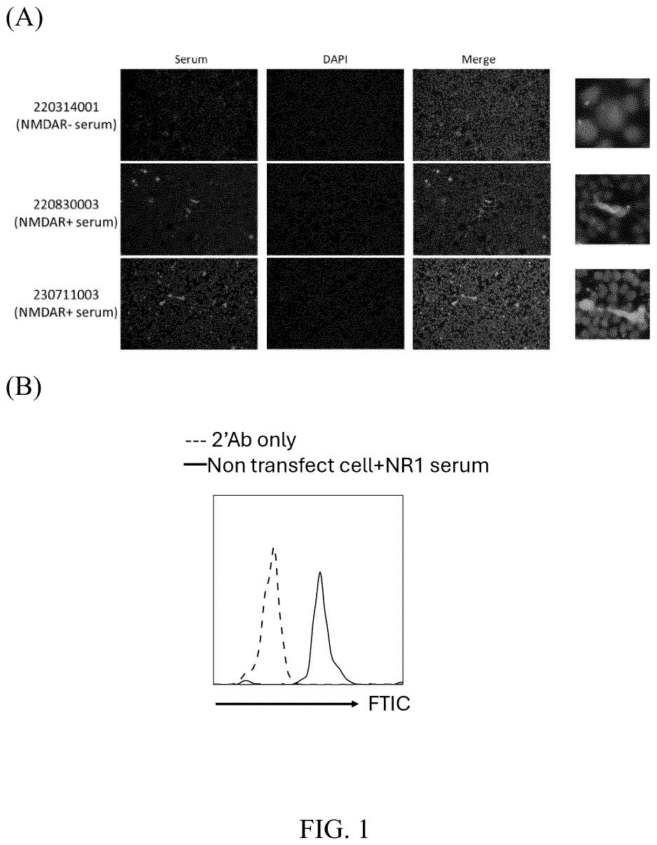

However, it was found that serum exhibited significant interfering signals that affected the interpretation of IFS reading and, worst, led to an overestimation of concentration in flow cytometry analysis. Further analysis revealed that most of the interfering signals seemed to originate from areas resembling cell nuclei. A nuclear stain (i.e., DAPI) was thus introduced into the IFS process to test this hypothesis. Results are depicted in FIG. 1.

As shown in FIG. 1, the interference signals originated from the serum almost completely overlapped with that of the nuclear signals, resulting in a shift of intensity peak in flow cytometry analysis.

1.3 Reducing the Non-Specific Interference Signals from Serum

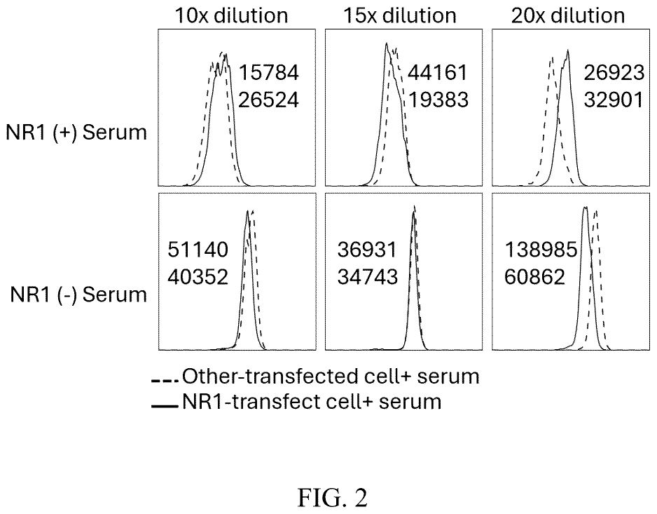

To solve the serum indued non-specific interfering signals exemplified in Example 1.2, serum samples were diluted 10-, 15- or 20-times before mixing with NR1 antigen-expressing cells; further, signals from cells transfected with control plasmids (i.e., plasmids that did not expressed NR1 antigens) were used as background noise and/or non-specific signals. Accordingly, signals from cells transfected with NR1 antigen-plasmids were subtracted by signals from cells transfected with control plasmids, and results are depicted in FIG. 2.

It was found that such approach could accurately isolate the real signal associated with NR1 expression, with serum sample being diluted 20-times gave the best result.

### 1.4 Detection of Autoantibodies in Serum Samples of AE Patients

Four serum samples from anti-NMDAR encephalitis patients, whose sample IDs were listed in Table 2, were used to demonstrate the use of the present antigen plasmids transfected cells in quantifying autoantibodies present in the serum samples of AE patients.

The concentrations of the anti-NMDAR autoantibody in the 4 serum samples were respectively 342.85 μg/mL, 144.73 μg/mL, incalculable and 162.60 g/mL calculated from a standard curve (Table 2). However, the anti-NMDAR autoantibody concentration for the sample number #220314001 determined by IFS was inconsistent with that determined by flow cytometry method. Thus, the data was re-calculated by using the improved method described in Example 1.3. After recalculation, the concentrations of the anti-NMDAR autoantibody in the four serum samples were respectively 14.23 μg/mL, 0.45 μg/mL, 0 μg/mL and 34.35 μg/mL, confirming the results from IF staining and flow cytometry (Table 3).

TABLE 2
The anti-NMDAR autoantibody concentration in four
AE serum samples calculated from a standard curve

| | | | Flow | Ab Conc |
|---|---|---|---|---|
| Sample ID # | IFS | Signals from NR1 | cytometry | (μg/mL) |
| 220830003 | + | 108453 | + | 342.85 |
| 230711003 | + | 53376 | + | 144.73 |
| 220314001 | − | 662000 | + | Incalculable |
| 231106002 | + | 59374 | + | 162.6 |

“+”: positive; “−”: negative.

TABLE 3
The anti-NMDAR autoantibody concentration in four AE
serum samples calculated by the method of Example 1.3

| | | Signals | | | | Ab |
|---|---|---|---|---|---|---|
| | | Control | | NR1 − | | Conc |
| Sample | | cells | NR1 | cells | Flow | (μg/ |
| ID # | IFS | (A) | (B) | (C = B − A) | cytometry | mL) |
| 220830003 | + | 102673 | 108453 | 5780 | + | 14.23 |
| 230711003 | + | 52486 | 53376 | 890 | + | 0.45 |
| 220314001 | − | 679000 | 662000 | −17000 | − | 0 |
| 231106002 | + | 46548 | 59374 | 12826 | + | 34.35 |

+: positive; −: negative.

The data in Table 3 confirmed that the improved method of Example 1.3 was useful for detecting and quantifying the autoantibody against neuronal antigen via flow cytometry.

### Example 2: Detection of Antibodies Against Neuronal Antigens in AE Patients by the Present Method

In this example, the present method was used to detect and quantify anti-NMDAR autoantibodies in serum and/or CSF samples collected from 14 subjects suspected of having AE with written informed consent according to similar procedures described in Example 1.3. Results are summarized in Table 4.

TABLE 4
Identification of anti-NMDAR autoantibodies in serum/CSF samples by the present method

| | | | | | | Flow cytometry | | |
|---|---|---|---|---|---|---|---|---|
| | | Type of | IFS | | | Ab Conc (μg/mL) | | |
| Patient # | Report ID # | sample | Visit 1 | Visit 2 | Visit 3 | Visit 1 | Visit 2 | Visit 3 |
| 1 | R20240703001 | CSF | + | + | | +8.01 | +6.136 | |
| | | serum | + | + | + | +4.808 | +4.564 | +2.6481 |
| 2 | R20240709001 | CSF | + | | | +1.49 | | |
| | | serum | + | | | +3.0 | | |
| | R20240905001 | serum | + | | | +0.4 | | |
| | R20241107002 | serum | − | | | −0 | | |
| | R20250123001 | serum | + | | | +0.29 | | |
| 3 | R20240813001 | serum | + | | | +20.48 | | |
| 4 | R20240813002 | serum | − | | | −0 | | |
| 5 | R20240813003 | serum | − | | | −0 | | |
| 6 | R20240813004 | CSF | + | | | +15.3 | | |
| | | serum | + | | | +13.2 | | |
| 7 | R20240905002 | CSF | + | | | +4.6458 | | |
| | R20240829001 | CSF | + | | | +3.744 | | |
| | R20250214003 | CSF | + | | | +0.358 | | |
| 8 | R20240829002 | CSF | − | | | −0 | | |
| 9 | R20241015001 | CSF | + | + | | +0.028 | +0.016 | |
| | R20241107001 | CSF | + | + | | +0.009 | +0.532 | |
| | R20241226008 | Serum | + | | | +0.2357 | | |
| | R20241226002 | CSF | + | | | +0.4534 | | |
| | | Serum | + | | | +0.06 | | |
| | R20241226003 | CSF | + | | | +0.00679 | | |
| | | Serum | + | | | +0.00000632 | | |
| | R20241226007 | CSF | + | | | +0.00344 | | |
| | | Serum | + | | | +0.0457 | | |
| | R20250214002 | Serum | + | + | | +0.30025 | +4.77891 | |
| 10 | R20241107003 | CSF | + | | | +1.453 | | |
| 11 | R20241226001 | CSF | + | | | +0.868 | | |
| 12 | R20241226004 | CSF | + | | | +2.5851 | | |
| | | Serum | + | | | +0.0000622 | | |
| 13 | R20241226005 | CSF | + | | | +0.2594 | | |
| | | Serum | + | | | +2.1126 | | |
| | R20241226006 | CSF | + | | | +0.2106 | | |
| | | Serum | + | | | +0.7146 | | |
| 14 | R20250214001 | Serum | + | | | +8903.654 | | |

“+”: positive; “−”: negative.

As summarized in Table 4, 4 subjects were identified to be healthy, while the other 10 subjects were determined to be AE subjects, due to positive identification of anti-NMDAR autoantibodies in serum and/or CSF. For AE subjects (i.e., patient #: 1, 2, 7 and 13), whose disease condition improved after treatment, as the concentration of anti-NMDAR autoantibodies in serum and/or CSF reduced in the following visits.

For patient #: 9, whose disease condition also showed improvement after treatment, as the concentration of anti-NMDAR autoantibodies in serum and/or CSF reduced in the following visits in 2024. However, the concentration of anti-NMDAR autoantibodies in serum rose up again in 2025.

In conclusion, the present disclosure provides novel methods of detecting and quantifying autoantibodies against neuronal antigens (including NMDAR, GABAR, AMPAR, Caspr2, LGI1 and DPPX). Based on the present methods, a skilled artisan may efficiently diagnose AE and/or the cause of AE based on the autoantibodies found in a biological sample of a subject, hence treatment may be timely administered to the subject to alleviate or ameliorate symptoms associated with AE.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of identifying and treating a subject having autoimmune encephalitis (AE) comprising:
(1) detecting an autoantibody against a neuronal antigen in a biological sample obtained from the subject by:
(a) transfecting a host cell with a plasmid comprising a nucleic acid encoding the neuronal antigen to produce a transfected host cell having the neuronal antigen expressed thereon;
(b) mixing the transfected host cell of step (a) and a control host cell having a first fluorophore labeled thereon to produce a mixture, in which the control host cell does not have the neuronal antigen thereon expressed thereon;
(c) adding the biological sample to the mixture of step (b), in which the biological sample has been diluted at least 15 times before adding to the mixture of step (b);
(d) adding an anti-human antibody to the product of step (c), wherein the anti-human antibody is conjugated to a second fluorophore.
(e) irradiating the products of step (d) independently with a first light having a first wavelength to excite the first fluorophore and a second light having a second wavelength to excite the second fluorophore;
(f) measuring respective signals emitted from the excited first and second fluorophores at a third and fourth wavelengths; and
(g) subtracting the signal measured at the third wavelength from the signal measured at the fourth wavelength thereby producing a real signal, which indicates the presence of the autoantibody in the biological sample;
wherein, steps (b) and (c) are conducted in the same vessel; and
(2) treating the subject by administering solumedrol or immunoglobulin (Ig) to the subject to ameliorate symptoms associated with AE.

2. The method of claim 1, further comprising after step (c) and before step (d),
(c-1) washing the product of step (c) with a buffer comprising 1% bovine serum albumin (BSA), 2 mM ethylenediaminetetraacetic acid (EDTA), and 0.02% sodium azide (NaN₃).

3. The method of claim 2, wherein in step (c), the biological sample has been diluted 20 times before adding to the mixture of step (b).

4. The method of claim 3, wherein steps (b) and (c) are conducted at 4° C. for 20 minutes.

5. The method of claim 1, wherein the first fluorophore is 2′-(4-ethoxyphenyl)-5-(4-methyl-1-piperazinyl)-2,5′-bi-1H-benzimidazole trihydrochloride, and the second fluorophore is fluorescein isothiocyanate (FITC).

6. The method of claim 1, wherein the biological sample is whole blood, plasma, serum, cerebrospinal fluid, or a biopsy taken from a human subject.

7. The method of claim 6, wherein the biological sample is serum.

8. The method of claim 6, wherein the biological sample is the cerebrospinal fluid.

9. The method of claim 1, wherein the host cell is human embryonic kidney (HEK) 293 cell.

10. The method of claim 1, wherein the neuronal antigen is N-methyl-D-aspartate receptor (NMDAR), α-amino-3-hydroxy-5-methyl-4-isoxazoleproprionic acid receptor (AMPAR), gamma-aminobutyric acid receptor (GABAR), contactin-associated protein-like 2 (Caspr2), leucine-rich glioma inactivated protein 1 (LGI1), or dipeptidyl-peptidase-like protein 6 (DPPX).

11. The method of claim 10, wherein the neuronal antigen is the NMDAR1, and the plasmid comprises the nucleic acid encoding the NMDAR1 having a polynucleotide sequence of SEQ ID NO: 1.

12. The method of claim 10, wherein the neuronal antigen is the GABAR, and the plasmid comprises the nucleic acid encoding the GABAR having a polynucleotide sequence of SEQ ID NO: 2.

13. The method of claim 10, wherein the neuronal antigen is the AMPAR, and the plasmid comprises the nucleic acid encoding the AMPAR having a polynucleotide sequence of SEQ ID NO: 3.

14. The method of claim 10, wherein the neuronal antigen is the Caspr2, and the plasmid comprises the nucleic acid encoding the Caspr2 having a polynucleotide sequence of SEQ ID NO: 4.

15. The method of claim 10, wherein the neuronal antigen is the LGI1, and the plasmid comprises the nucleic acid encoding the LGI1 having a polynucleotide sequence of SEQ ID NO: 5.

16. The method of claim 10, wherein the neuronal antigen is the DPPX, and the plasmid comprises the nucleic acid encoding the DPPX having a polynucleotide sequence of SEQ ID NO: 6.

17. The method of claim 1, wherein in step (1), further comprises quantifying the autoantibody in the biological sample by:
(i) providing a control antibody specific to the neuronal antigen, wherein the control antibody is conjugated to the second fluorophore;
(ii) serially diluting the control antibody to produce a plurality of diluted antibodies respectively having pre-determined concentrations;
(iii) respectively irradiating the plurality of diluted antibodies of step (ii) with the light having the second wavelength;
(iv) measuring the intensities of respective signals emitted from the diluted antibodies of step (iii) at the fourth wavelength;
(v) constructing a calibration plot of the concentrations of the plurality of diluted antibodies as a function of the intensities of the signals measured in step (iv); and
(vi) interpolating from the calibration plot of step (v) of the concentration of the autoantibody based on the intensity of the real signal produced in step (g).

18. The method of claim 1, wherein in step (2), further comprising administering rituximab, cyclophosphamide or both to the subject.
